(19) United States
(12) Patent Application Publication
(10) Pub. No.: US 2021/0128441 A1
(43) Pub. Date: May 6, 2021

(54) COMPOSITIONS AND METHODS FOR CURLY HAIR

(71) Applicant: L'OREAL, Paris (FR)

(72) Inventors: Seyma ASLAN, Clifton, NJ (US); Rita CHOKSHI, Monroe Township, NJ (US); Alina RODRIGUEZ, New Haven, CT (US); Jonathan Jiahao SUN, New Haven, CT (US)

(21) Appl. No.: 17/085,312

(22) Filed: Oct. 30, 2020

Related U.S. Application Data
(60) Provisional application No. 62/929,033, filed on Oct. 31, 2019.

(51) Int. Cl. A61K 8/73; A61K 8/97; A61K 8/34; A61K 8/898; A61K 8/92; A61K 8/41; A61Q 5/06

(52) U.S. Cl. CPC A61K 8/731; A61K 8/97; A61K 8/342; A61K 8/898; A61K 2800/59; A61K 8/416; A61Q 5/06; A61K 2800/41; A61K 8/922

(57) ABSTRACT

The present disclosure relates to compositions and methods for treating hair, and for improving the appearance and/or feel of curly hair. The compositions comprise a combination of nanocrystalline cellulose and murumuru butter, and the methods comprise applying the compositions to the hair.

### TECHNICAL FIELD

The present disclosure relates to compositions and methods for treating keratin fibers, such as hair, and to compositions and methods for improving the appearance and/or feel of curly hair.

### BACKGROUND

Many people are dissatisfied with the appearance of their hair, and desire to treat their hair in order to improve the appearance and/or feel thereof. For example, while people who have curly hair may wish to maintain the curls, they may also desire to gain better curl definition and/or regularity and/or to improve curl retention, and/or may seek a more natural look and feel of their curly hair. Attributes that consumers desire include, for example, soft (i.e. not “crunchy” feeling) and non-frizzy curls, curls which last and hold overnight, and which are moisturized but not greasy-feeling. Further, consumers desire products which use natural ingredients.

As such, there is a consumer desire for methods to treat keratin fibers, in particular human keratin fibers such as curly hair, which may make it possible to maintain curls while obtaining good curl definition and/or regularity, better movement, good volume, frizz control, and/or natural shape, and which may be long-lasting, non-irritating, and/or non-toxic.

The present disclosure therefore addresses these concerns and relates to compositions which surprisingly provide a synergistic benefit of combinations of various components, which unexpectedly provides improved properties of appearance and/or feel to curly hair, and methods of using such compositions to improve the appearance and/or feel of curly hair.

### SUMMARY

The present disclosure relates to compositions and methods for treating hair, and for improving the appearance and/or feel of curly hair.

According to one embodiment, the disclosure relates to cosmetic compositions comprising nanocrystalline cellulose, murumuru butter, at least one fatty alcohol, at least one aminosilicone, at least one oil, and at least one cationic surfactant.

In further embodiments, the disclosure relates to cosmetic compositions comprising nanocrystalline cellulose in an amount ranging from about 0.1% to about 3%, murumuru butter in an amount ranging from about 0.1% to about 5%, at least one fatty alcohol in an amount ranging from about 3% to about 8%, at least one aminosilicone in an amount ranging from 0.01% to about 2%, at least one oil in an amount ranging from about 0.1% to about 3%, and at least one cationic surfactant in an amount ranging from about 1% to about 5%, all amounts relative to the total weight of the composition.

In yet further embodiments, the disclosure relates to methods of treating hair comprising applying to the hair a cosmetic composition comprising nanocrystalline cellulose, murumuru butter, at least one fatty alcohol, at least one aminosilicone, at least one oil, and at least one cationic surfactant.

In still further embodiments, the disclosure relates to methods of treating hair comprising applying to the hair a cosmetic composition comprising nanocrystalline cellulose in an amount ranging from about 0.1% to about 3%, and murumuru butter in an amount ranging from about 0.1% to about 5%, relative to the total weight of the composition.

### FIGURE

FIG. 1 shows a comparison between swatches of curly hair treated with a composition according to the disclosure compared to two compositions not within the disclosure.

### DESCRIPTION

The present disclosure relates to compositions and methods for improving the appearance and feel of curly hair. The compositions comprise a synergistic combination of nanocrystalline cellulose and murumuru butter. The methods comprise applying the compositions to the hair.

### I. Compositions

The compositions according to the disclosure comprise a synergistic combination of nanocrystalline cellulose and murumuru butter, which combination surprisingly and unexpectedly leads to improved properties of the hair such as softer, smoother, more defined curls, longer curl retention, good curl pick-up with springiness, closed ends, and/or good shape. The compositions may, for example, be leave-on hair compositions.

### Nanocrystalline Cellulose

Cellulose is highly abundant in nature and is a primary constituent of plants. Nanocrystalline cellulose is a crystalline portion of cellulose which may be formed by acid hydrolysis of cellulose combined with mechanical treatment. These nanometer size cellulose particles are typically crystalline in nature, insoluble in water, stable, chemically inactive, and physiologically inert. Nanocrystalline cellulose may be in the form of “nanocrystals” which are substantially spherical, which may be in the shape of fibers, plates, spindles, needles, etc., or which may be a combination thereof. It should be understood that the use of the term “nanocrystalline cellulose” as used herein is intended to include “nanocellulose,” “nanocrystalline cellulose,” “cellulose nanocrystals,” “cellulose whiskers,” “cellulose nanofibers,” and variations thereof, without limitation. As a non-limiting example, nanocrystalline cellulose prepared as described in WO2016/015148A1, the disclosure of which is incorporated by reference, may be used. The nanocrystalline cellulose useful according to the disclosure may optionally be chemically-modified.

In various embodiments, nanocrystalline cellulose having substantially spherical nanocrystals with an average particle size of less than about 25 μm, less than about 20 μm, less than about 15 μm, less than about 10 μm, less than about 5 μm, less than about 1 μm, less than about 500 nm, less than about 250 nm, less than about 200 nm, or less than about 100 nm, may be chosen. For example, the nanocrystals may have an average particle size ranging from about 50 nm to about 20 μm, such as about 50 nm to about 20 μm, about 50 nm to about 15 μm, about 50 nm to about 10 μm, about 50 nm to about 5 μm, about 50 nm to about 1 μm, about 50 nm to about 500 nm, about 100 nm to about 500 nm, or about 50 nm to about 200 nm. The average particle size is the particle size distribution (D₅₀), also known as the median diameter or the medium value of the particle size distribution, which is the value of the particle diameter at 50% in the cumulative distribution.

As a further example, nanocrystalline cellulose having nanocrystals that are in the shape of fibers, spindles, plates, needles, etc., with an average length of less than about 25 μm, less than about 20 μm, less than about 15 μm, less than about 10 μm, less than about 5 μm, less than about 1 μm, less than about 500 nm, less than about 250 nm, less than about 200 nm, or less than about 100 nm, may be chosen. For example, the nanocrystals may have an average length ranging from about 50 nm to about 10 μm, such as about 50 nm to about 5 μm, about 50 nm to about 1 μm, about 50 nm to about 500 nm, about 100 nm to about 500 nm, or about 50 nm to about 200 nm. In certain embodiments, the average length is about 100 nm to about 450 nm, such as about 150 nm to about 450 nm, or about 200 nm to about 400 nm.

In various embodiments, the nanocrystalline cellulose nanocrystals may have an average width (diameter) of less than about 2 μm, less than about 1 μm, less than less than about 500 nm, less than about 250 nm, less than about 100 nm, less than about 50 nm, less than about 25 nm, less than about 20 nm, less than about 15 nm, or less than about 10 nm. For example, the nanocrystals may have an average width ranging from about 5 nm to about 1 μm, such as about 5 nm to about 1 μm, about 5 nm to about 500 nm, about 5 nm to about 100 nm, about 5 nm to about 50 nm, or about 5 nm to about 20 nm. In certain embodiments, the average width is about 1 nm to about 25 nm, such as about 5 nm to about 20 nm, about 10 nm to about 20 nm, or about 5 nm to about 10 nm.

It is well within the ability of those skilled in the art to determine the shape and/or average particle size of the nanocrystals. For example, the average particle size or particle size distribution may be determined by laser diffraction granulometry or Scanning Electron Microscopy (SEM), among other known methods.

The nanocrystalline cellulose may, in various embodiments, comprise linear, long-chain glucose polymers rich in oxygen, such as hydroxyl groups. The nanocrystalline cellulose may optionally also comprise acidic groups attached to the surface. Further, the nanocrystalline cellulose may optionally be charged.

In various embodiments, the nanocrystalline cellulose may be present in the composition in an amount ranging up to about 10%, such as up to about 9%, up to about 8%, up to about 7%, up to about 6%, up to about 5%, up to about 4%, up to about 3%, up to about 2%, up to about 1%, or up to about 0.5%, relative to the total weight of the composition. For example, the nanocrystalline cellulose may be present in an amount ranging from about 0.001% to about 10%, from about 0.01% to about 8%, from about 0.1% to about 6%, from about 0.2% to about 4%, such as about 0.2% to about 2%, relative to the total weight of the composition. In yet further examples, the nanocrystalline cellulose may be present in an amount ranging from about 0.001% to about 5%, from about 0.01% to about 4%, from about 0.1% to about 3%, from about 0.15% to about 2%, from about 0.2% to about 1.5%, or from about 0.25% to about 1%, relative to the total weight of the composition.

In one embodiment, the nanocrystalline cellulose is present in an amount of about 0.1%; in a further embodiment, the nanocrystalline cellulose is present in an amount of about 0.25%; in another embodiment, the nanocrystalline cellulose is present in an amount of about 0.5%; in yet a further embodiment, the nanocrystalline cellulose is present in an amount of about 0.75%; in a still further embodiment, the nanocrystalline cellulose is present in an amount of about 1%, all weights relative to the total weight of the composition.

### Murumuru Butter

The compositions according to the disclosure comprise murumuru butter. Astrocaryum murumuru is a palm plant which may, for example, be found in Brazil; murumuru butter is extracted from the seeds of the plant.

The murumuru butter may be present in the compositions in an amount ranging up to about 10%, such as up to about 9%, up to about 8%, up to about 7%, up to about 6%, up to about 5%, up to about 4%, up to about 3%, up to about 2%, up to about 1.5%, or up to about 1%, relative to the total weight of the composition. For example, the murumuru butter may be present in an amount ranging from about 0.001% to about 10%, from about 0.01% to about 8%, from about 0.1% to about 6%, or from about 0.5% to about 4%, relative to the total weight of the composition. In yet further examples, the murumuru butter may be present in an amount ranging from about 0.001% to about 5%, from about 0.01% to about 4%, from about 0.1% to about 3%, from about 0.25% to about 3%, from about 0.5% to about 2.5%, from about 0.5% to about 2%, from about 1% to about 2%, or from about 1% to about 1.5%, relative to the total weight of the composition.

In one embodiment, the murumuru butter is present in an amount of about 0.75%; in a further embodiment, the murumuru butter is present in an amount of about 1%; in another embodiment, the murumuru butter is present in an amount of about 1.25%; in another embodiment, the murumuru butter is present in an amount of about 1.5%; in yet a further embodiment, the murumuru butter is present in an amount of about 1.75%; in a still further embodiment, the murumuru butter is present in an amount of about 2%, all weights relative to the total weight of the composition. Optionally, the amounts of nanocrystalline cellulose and murumuru butter may be chosen so that the ratio of nanocrystalline cellulose:murumuru butter ranges from about 1:10 to about 10:1. For example, the ratio may range from about 1:8 to about 8:1, about 1:6 to about 6:1, about 1:5 to about 5:1, about 1:4 to about 4:1, about 1:3 to about 3:1, about 1:2 to about 2:1, or about 1:1.

### Additional Components

Additional hair treatment components may also be included in the compositions according to the disclosure. Although those skilled in the art will be careful to ensure the synergistic combination of nanocrystalline cellulose and murumuru butter is not substantially interfered with, it is well within the ability of the skilled artisan to choose additional hair treatment components for preparing compositions according to the disclosure. For example, such components may include fatty compounds, conditioning agents, humectants, solvents, surfactants, aminosilicones, preservatives, fragrances, plant extracts, vitamins, etc.

The compositions according to the disclosure include at least one solvent, optionally chosen from water and cosmetically acceptable organic solvents, as well as mixtures thereof. The total amount of solvent can vary, but typically ranges from about 15% to about 95%, based on the total weight of the composition. For example, the total amount of solvent may range from about 20% to about 95%, about 30% to about 95%, about 40% to about 95%, about 50% to about 95%, about 60% to about 95%, about 70% to about 95%, about 80% to about 95%, about 80% to about 90%, or about 85% to about 90% all weights relative to the total weight of the composition. By way of example only, the solvent may be chosen from water, glycerin, and a mixture of water and glycerin.

The compositions described herein may optionally contain at least one cationic surfactant. The term “cationic surfactant” means a surfactant that is positively charged when it is contained in the composition according to the disclosure. This surfactant may bear one or more positive permanent charges or may contain one or more functions that are cationizable in the composition according to the disclosure. Non-limiting examples of useful cationic surfactants include behentrimonium chloride, cetrimonium chloride, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide. Mixtures of cationic surfactants may also be used.

The at least one cationic surfactant may be present in an amount ranging up to about 10%, such as up to about 9%, up to about 8%, up to about 7%, up to about 6%, up to about 5%, up to about 4%, up to about 3.5%, up to about 3%, up to about 2.5%, up to about 2%, up to about 1.5%, up to about 1%, or up to about 0.5%, relative to the total weight of the composition. For example, the at least one surfactant may be present in an amount ranging from about 0.001% to about 10%, from about 0.01% to about 8%, from about 0.1% to about 6%, or from about 0.5% to about 4%, relative to the total weight of the composition. In yet further examples, the at least one surfactant may be present in an amount ranging from about 0.001% to about 5%, from about 0.01% to about 5%, or from about 0.1% to about 5%, relative to the total weight of the composition.

In further embodiments, the at least one cationic surfactant may be present in the composition in an amount of at least 0.1%. For example, the at least one cationic surfactant may be present in the composition in an amount ranging from about 0.1% to about 10%, such as about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, or about 0.1% to about 1%, relative to the total weight of the composition. In further embodiments, the at least one cationic surfactant may be present in the composition in an amount ranging from about 0.2% to about 5%, such as about 02% to about 4%, about 0.2% to about 3%, about 0.2% to about 2%, about 0.2% to about 1%, about 0.3% to about 5%, such as about 0.3% to about 4%, about 0.3% to about 3%, about 0.3% to about 2%, about 0.3% to about 1%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2.5%, or about 0.5% to about 2%, relative to the total weight of the composition.

The compositions may also optionally comprise at least one fatty compound. As used herein, “fatty compound” means an organic compound insoluble in water at normal temperature (25° C.) and at atmospheric pressure (760 mmHg) (solubility below 5% and such as below 1% and further such as below 0.1%. Fatty compounds are generally soluble in organic solvents in the same conditions of temperature and pressure.

In certain embodiments, the at least one fatty compound may be chosen from lower alkanes, fatty alcohols, esters of fatty acid, esters of fatty alcohol, oils such as mineral, vegetable, animal, silicone and non-silicone oils, and silicone and non-silicone waxes. The term “oil” means any fatty substance which is in liquid form at room temperature (20-25° C.) and at atmospheric pressure (760 mmHg). Oils that are suitable for use herein may be volatile or non-volatile. The term “volatile oil” relates to oil that is capable of evaporating on contact with the keratin fiber in less than one hour, at room temperature and atmospheric pressure. The volatile oil(s) are liquid at room temperature and have a non-zero vapor pressure, at room temperature and atmospheric pressure, ranging in particular from 0.13 Pa to 40 000 Pa (10⁻³ to 300 mmHg). The term “non-volatile oil” relates to oil which remains on the keratin fiber, at room temperature and atmospheric pressure, for at least several hours and which in particular has a vapor pressure of less than 10⁻³ mmHg (0.13 Pa).

The cosmetic compositions described herein may comprise one or more silicone oils. The term “silicone oil” relates to oil comprising at least one silicon atom, and especially at least one Si—O group. Non-limiting examples of silicone oils include dimethicone, cyclomethicone, polysilicone-11, phenyl trimethicone, trimethylsilylamodimethicone, and stearoxytrimethylsilane. In some cases, the cosmetic composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Other examples of silicone oils that may be mentioned include volatile linear or cyclic silicone oils, especially those with a viscosity 8 centistokes (8×10⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The cosmetic compositions described herein may comprise one or more fluoro oils. The term “fluoro oil” relates to oil comprising at least one fluorine atom. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

The cosmetic compositions described herein may comprise one or more hydrocarbon-based oils. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil or a synthetic hydrocarbon-based oil. Further, suitable oil may be a mineral hydrocarbon-based oil, a plant hydrocarbon-based oil, or a synthetic hydrocarbon-based oil.

For example, the hydrocarbon-based oil may be a saturated hydrocarbon, an unsaturated hydrocarbon, lipids, triglycerides, a natural oil, and/or a synthetic oil. In some embodiments, the compositions include a synthetic oil selected from the group consisting of hydrogenated polyisobutene and hydrogenated polydecene.

The hydrocarbon-based oil may be a non-volatile hydrocarbon-based, such as:

(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil;

(ii) synthetic ethers containing from 10 to 40 carbon atoms;

(iii) linear or branched hydrocarbons of mineral or synthetic origin, such as petroleum jelly, polydecenes, hydrogenated polyisobutene such as Parleam, and 4 0 squalane;

(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is 10, for instance Purcellin oil (cetearyl octanoate), isopropyl myristate, isopropyl palmitate, C12-C15 alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI® by Enichem Augusta Industriale, and also di(linear C14-C15 alkyl) tartrates, such as those sold under the name Cosmacol ETL® by the same company; or acetates;

(v) fatty alcohols, such as those that are liquid at room temperature, e.g. containing a branched and/or unsaturated carbon-based chain containing from 12 to 26 carbon atoms, for instance octyldodecanol, cetearyl alcohol, stearyl alcohol, isostearyl alcohol, oleyl alcohol, 2-hexyldecanol, 2-butyloctanol or 2-undecylpentadecanol;

(vi) higher fatty acids, such as oleic acid, linoleic acid or linolenic acid;

(vii) carbonates, such as dicaprylyl carbonate, such as the product sold under the name Cetiol CC® by Cognis;

(viii) fatty amides, such as isopropyl N-lauroyl sarcosinate, such as the product sold under the trade name Eldew SL 205® from Ajinomoto; and

(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang.

In certain instances, the non-volatile hydrocarbon-based oils are glyceride triesters such as caprylic/capric acid triglycerides, synthetic esters such as isononyl isononanoate, oleyl erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C8-C16 esters, and isohexyl neopentanoate.

When present, the at least one oil is present in the composition in an amount ranging up to about 10%, such as up to about 9%, up to about 8%, up to about 7%, up to about 6%, up to about 5%, up to about 4%, up to about 3%, up to about 2%, or up to about 1%, all weights relative to the total weight of the composition. By way of example, the at least one oil may be present in an amount ranging from about 0.01% to about 10%, such as about 0.01% to about 5%, about 0.01% to about 4%, about 0.01% to about 3%, about 0.01% to about 2%, about 0.1% to about 5%, about 0.1% to about 4%, about 0.1% to about 3%, about 0.1% to about 2%, about 1% to about 4%, about 1% to about 3%, or about 1% to about 2%.

The compositions preferably comprise at least one fatty alcohol. In at least certain embodiments, “fatty alcohol” refers to any alcohol with a carbon chain of C5 or greater, such as, for example, C8 or greater, C10 or greater, and C12 or greater. The at least one fatty alcohol may be chosen from, for example, C9-C11 alcohols, C12-C13 alcohols, C12-C15 alcohols, C12-C16 alcohols, C14-C15 alcohols, arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, and tridecyl alcohol.

The fatty alcohols usable as fatty substances in the composition of the disclosure include, but are not limited to, those that are alkoxylated or non-alkoxylated, saturated or unsaturated, linear or branched, and have from 6 to 30 carbon atoms and more particularly from 8 to 30 carbon atoms. For example, cetyl alcohol, stearyl alcohol and their mixture (cetylstearyl alcohol or cetearyl alcohol), octyldodecanol, 2-butyloctanol, 2-hexyldecanol, 2-undecylpentadecanol, oleic alcohol, or linoleic alcohol may be chosen.

As used herein, “alkoxylated fatty alcohol” refers to any fatty alcohol with a carbon chain of C5 or greater, as defined above, further comprising at least one alkoxy group. For example, the at least one alkoxylated fatty alcohol may have a carbon chain of C8 or greater, C10 or greater, and C12 or greater. Further, for example, the at least one alkoxylated fatty alcohol may be chosen from alkoxylated polymers (including co-, ter- and homo-polymers) derived from alcohols such as glycerol (e.g. polyglyceryl derived from four glycerol molecules). The at least one alkoxy group of the at least one alkoxylated fatty alcohol may, for example, be derived from an alkoxylation reaction carried out with alkylene oxide. Non-limiting examples of at least one alkoxylated fatty alcohol include any fatty alcohol comprising at least one polyethylene glycol ether and any fatty alcohol comprising at least one polypropylene glycol ether.

Non-limiting examples of the at least one alkoxylated fatty alcohol include ceteareth-2, ceteareth-3, ceteareth-4, ceteareth-5, ceteareth-6, ceteareth-7, ceteareth-8, ceteareth-9, ceteareth-10, ceteareth-1, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, laureth-1, laureth-2, laureth-3, laureth-4, laureth-5, laureth-6, laureth-7, laureth-8, laureth-9, laureth-10, laureth-1 1, laureth-12, laureth-13, laureth-14, laureth-15, lauretih-16, laureth-20, laureth-23, laureth-25, laureth-30, laureth-40, deceth-3, deceth-5, oleth-5, oleth-30, steareth-2, steareth-10, steareth-20, steareth-100, cetylsteareth-12, ceteareth-5, ceteareth-5, polyglyceryl 4-lauryl ether, polyglyceryl 4-oleyl ether, polyglyceryl 2-oleyl ether, polyglyceryl 2-cetyl ether, polyglyceryl 6-cetyl ether, polyglyceryl 6-oleylcetyl ether, polyglyceryl 6-octadecyl ether, C9-C11 pareth-3, C9-C11 pareth-6, C11-C15 pareth-3, C11-C15 pareth-5, C11-C15 pareth-12, C11-C15 pareth-20, C12-C15 pareth-9, C12-C15 pareth-12, and C22-C24 pareth-33.

When present, the at least one fatty alcohol may be present in an amount up to about 15%, such as up to about 10%, up to about 9%, up to about 8%, up to about 7%, up to about 6%, up to about 5%, up to about 4%, up to about 3%, up to about 2%, or up to about 1%, all weights relative to the total weight of the composition. By way of example, the at least one fatty alcohol may be present in an amount ranging from about 0.1% to about 15%, such as about 1% to about 10%, about 2% to about 9%, about 3% to about 8%, about 4% to about 7%, about 5% to about 7%, about 5.5% to about 6.5%, or about 6%.

The compositions may optionally contain at least one conditioning agent. By way of non-limiting example, the at least one conditioning agent may be chosen from amino acids, proteins, extracts, fats, oils, esters, transesters, hydrocarbons, quats, polyquats, zwitterionic surfactants, amphoteric surfactants, alcohols, polyols, humectants, alkanolamides, fatty acids, ketones, and mixtures thereof. In various embodiments, the conditioning agent is present in an amount from about 0.001% to about 10% by weight, based on the weight of the composition, such as from about 0.001% to about 5% by weight, based on the weight of the composition.

Non-limiting examples of conditioning agents include Arginine, Asparagine, Aspartic Acid, Carnitine, Cocoyl sarcosine, Glycine, Glutamic acid, Histidine, Hydroxyproline, Acetyl Hydroxy praline, Isoleucine, Lysine, Lauroyl Lysine, Lauroyl Sarcosine, Methionine, Phenylalanine, Polylysine, Potassium Cocoyl Glutamate, Proline, Sarcosine, Serine, Rice amino acids, Silk amino acids, Wheat amino acids, Sodium Glutamate, Sodium Lauroyl Glutamate, Sodium PCA, Stearoyl sarcosine, Threonine, Tyrosine, Tryptophan, Valine, Casein, Collagen, Procollagen, Gelatin, Keratin, Glycoproteins, Hydrolyzed wheat protein, Hydrolyzed soy protein, Hydrolyzed oat protein, Hydrolyzed rice protein, Hydrolyzed vegetable protein, Hydrolyzed yeast protein, Whey protein, Ginkgo Biloba Nut extract, Salix Alba (Willow) Bark Extract, Morus Alba (Mulberry) Leaf, Behentrimonium Chloride, Behenamidopropyl PG-Dimonium Chloride, Behentrimonium Methosulfate, Cocotrimonium Methosulfate, Olealkonium Chloride, Steartrimonium Chloride, Babassuamidopropalkonium Chloride, Hydroxypropyl Guar, Hydroxypropyltrimonium chloride, Laurdimonium Hydroxypropyl Hydrolyzed Soy Protein, Steardimonium Hydroxypropyl Hydrolyzed Wheat Protein, Quaternium-22, Quaternium-27, Quaternium-87, Polyquaternium-4, Polyquaternium-6, Polyquaternium-10, Polyquaternium-11, Polyquaternium-44, Polyquaternium-67, Silicone Quaterium-8, Amodimethicone, Aminopropyldimethicone, Phenyltrimethicone, Cyclomethicone, Dimethicone, Hexyl Dimethicone, Dilinoleamidopropyl Dimthylamine Dimethicone PEG-7 Phosphate, C26-28 Alkyl Dimethicone, PEG-8 Dimethicone, PPG-12 Dimethicone, Polysilicone-13, Trideceth-9 PG-Amodimethicone, Bis-PEG-12 Dimethicone Beeswax, Capric/Caprylic Triglyceride, Petrolatum, Mineral Oil, Lanolin Oil, Cocos nucifera (Coconut) Oil, Olea Europea (Olive) Fruit Oil, Simmondsia Chinensis (Jojoba) Seed Oil, Prunus Armeniaca (Apricot) Kernel Oil, Crambe Abyssinica Seed Oil, Vegetable Oil, Zea Mays (Corn) Oil, Acetylated Lanolin Alcohol, Cetearyl Isononanoate, Cetearyl Ethylhexanoate, Cetearyl Palmitate, Hydrogenated Olive Oil Hexyl Esters, Triethylhexanoin, Ceramide-3, Caprylyl Glycol, Cetyl Glycol, Glycerin, Panthenol, Phytantriol, Methanediol, Inositol, PPG-35-Buteth-45, PPG-5 Butyl Ether, Cocoamidopropyl Betaine, Coco-Betaine, Cocoamidopropyl Hydroxysultaine, Lauramidopropyl Betaine, Lauryl Betaine, Oleamidopropyl Betaine, Disodium Cocoamphodiacetate, Disodium Cocoamphodipropionate, Disodium Lauroamphodiacetate, Sodium Cocoamphopropionate, Sodium Cocoamphoacetate, Acetamide MEA, Behenamide MEA, Linoleamide DEA, Linoleamide MEA, Linoleamide MIPA, Linoleic Acid, Linolenic Acid, Maltodextrin, Niacin, Polyacrylate-1 Crosspolymer, Polyester-4, Pyridoxine HCl, Phytosphingosine, Salicylic Acid, Squalane, Squalene, Thiodiglycoamide, Zinc Pyrithione, and mixtures thereof.

The compositions described herein may optionally contain at least one humectant. Humectants are hygroscopic compounds which help to retain moisture in the hair. In one or more embodiments, the humectant may be selected from the group consisting of glycerin, propylene glycol (propane diol), sorbitol, butylene glycol, ethylene glycol, polyethylene glycols having from 4 to 250 repeating ethylene glycol units, ethoxydiglycol, and mixtures thereof.

If present, at least one humectant may be present in the composition in an amount ranging up to about 5%, such as up to about 4%, up to about 3%, up to about 2%, up to about 1%, up to about 0.5%, up to about 0.25%, up to about 0.1%, up to about 0.01%, or up to about 0.001%, relative to the total weight of the composition. For example, the at least one humectant may be present in an amount ranging from about 0.001% to about 5%, from about 0.001% to about 1%, from about 0.005% to about 0.5%, from about 0.005% to about 0.1%, or about 0.01%, relative to the total weight of the composition.

The compositions may optionally comprise at least one aminosilicone. Exemplary, non-limiting aminosilicones include those chosen from aminosilicones of formula (I):

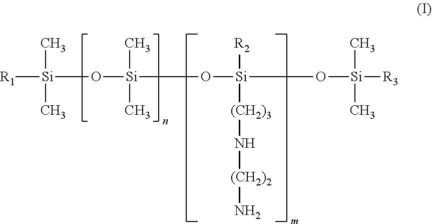

wherein:
m and n are numbers such that the sum (n+m) ranges, for example, from 1 to 2000, such as from 50 to 250 and further such as from 100 to 200,
n is a number ranging from 0 to 999, such as from 49 to 249 and further, such as from 125 to 175, and m is a number ranging from 1 to 1000, such as from 1 to 10 and further such as from 1 to 5; and
R₁, R₂ and R₃, which may be identical or different, are chosen from hydroxyl and C₁-C₄ alkoxy radicals, wherein at least one of the radicals R₁, R₂ and R₃ is chosen from C₁-C₄ alkoxy radicals.

In one embodiment, the alkoxy radical is a methoxy radical.

The hydroxyl/alkoxy molar ratio may range, for example, from 0.2:1 to 0.4:1 and further, for example, from 0.25:1 to 0.35:1, and even further, for example, is equal to 0.3:1.

The aminosilicone of formula (I) can have a weight-average molecular mass ranging, for example, from 2,000 to 1,000,000, and further, for example, from 3,500 to 200,000.

In other embodiments, further non-limiting exemplary aminosilicones include those chosen from aminosilicones of formula (II):

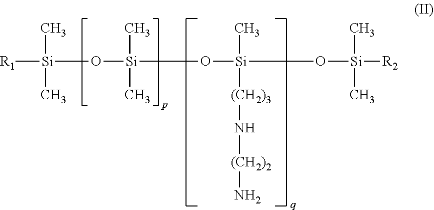

wherein:
p and q are numbers such that the sum (p+q) ranges, for example, from 1 to 2000, such as from 50 to 350 and further such as from 150 to 250,
p is a number ranging from 0 to 999, and further, for example, from 49 to 349, and even further, for example, from 159 to 239; and q is a number ranging from 1 to 1000, and further, for example, from 1 to 10 and even further, for example, from 1 to 5; and
R₁, R₂, which may be identical or different, are chosen from hydroxyl and C₁-C₄ alkoxy radicals, wherein at least one of the radicals R₁ and R₂ is chosen from C₁-C₄alkoxy radicals.

In one embodiment, the alkoxy radical is a methoxy radical.

The hydroxyl/alkoxy molar ratio may range, for example, from 1:0.8 to 1:1.1 and further, for example, from 1:0.9 to 1:1, and even further, for example, is equal to 1:0.95.

The aminosilicone of formula (II) may have a weight-average molecular mass ranging, for example, from 2,000 to 200,000, and further, for example, from 5,000 to 100,000, and even further, for example, from 10,000 to 50,000.

The commercial products corresponding to the aminosilicones of formula (I) or (II) may include in their composition at least one other aminosilicone whose structure is different from the formulae (I) and (II).

In various embodiments, the aminosilicone may be chosen from amodimethicones.

A product containing aminosilicones of formula (I) is that by the company Wacker under the name Belsil ADM 652®.

Products containing aminosilicones of formula (II) are those by the company Wacker under the names Fluid WR 1300®.

If present, the at least one aminosilicone may be present in the composition in an amount of at least 0.01%. For example, the at least one aminosilicone may be present in the composition in an amount ranging from about 0.01% to about 10%, such as about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, or about 0.01% to about 1%, relative to the total weight of the composition. In further embodiments, the at least one aminosilicone may be present in the composition in an amount ranging from 0.1% to about 5%, such as about 0.1% to about 4%, about 0.1 to about 3%, about 0.1% to about 2%, about 0.1% to about 1%, about 0.2% to about 10%, about 0.2% to about 9%, about 0.2% to about 8%, about 0.2% to about 7%, about 0.2% to about 6%, about 0.2% to about 5%, about 0.2% to about 4%, about 0.2% to about 3%, about 0.2% to about 2.5%, about 0.2% to about 2%, about 0.2% to about 1%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2.5%, or about 0.5% to about 2%, relative to the total weight of the composition.

The composition optionally comprises at least one preservative. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, ethylenediamine-tetraacetic acid (EDTA), potassium sorbate, and/or grapefruit seed extract, or combinations thereof. More than one preservative may be included in the composition. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol).

If present, the preservative(s) may be present in the composition in an amount ranging from about 0.01% to about 5% by weight, such as about 0.01% to about 3%, about 0.01% to about 1%, or about 0.01% to about 0.5% by weight of the composition.

Additional components may also optionally be included. For example, compositions according to the invention may comprise components such as fragrances, pigments or colorants, chelating agents, softeners, antioxidants, opacifiers, stabilizers, moisturizing agents, vitamins, polymers, thickening agents, extracts, or any other ingredient useful in hair treatment compositions. Such components may be included in or excluded from the hair treatment compositions depending on the specific combinations and amounts of other components, the form of the compositions (e.g., hair spray, cream, paste, conditioner, etc.), and/or the intended use of the formulation.

If present, the additional optional component(s) may be present in the composition in an amount ranging from about 0.01% to about 5% by weight, such as about 0.01% to about 3%, about 0.01% to about 1%, or about 0.01% to about 0.5% by weight of the composition.

### II. Methods

The methods according to the disclosure comprise treating keratinous fibers with the compositions described herein. For example, the hair may optionally be shampooed, optionally conditioned, and optionally dried, after which a composition according to the disclosure may be applied to wet, damp, or dry hair. If wet, the hair may be allowed to air dry with the composition on the hair, or the hair may be dried with the aid of a hair drying apparatus such as a hair dryer or diffuser. The hair may optionally be styled with a composition according to the disclosure on the hair, for example while the hair is wet or damp, or after it dries with the composition thereon. In various embodiments, the compositions are considered to be leave-on hair styling compositions, and thus, the compositions may be applied to the hair and left on for several hours to several days, or as long as the style is desired to be retained.

It is to be understood that all definitions herein are provided for the present disclosure only.

It to be understood that, as used herein the terms “the,” “a,” or “an,” mean “at least one,” and should not be limited to “only one” unless explicitly indicated to the contrary. Thus, for example, reference to “a preservative” includes examples having two or more preservatives, unless the context clearly indicates otherwise.

Unless otherwise expressly stated, it is in no way intended that any method set forth herein be construed as requiring that its steps be performed in a specific order. Accordingly, where a method does not expressly recite an order to be followed by its steps or it is not otherwise specifically stated in the claims or description that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method that comprises A+B+C include embodiments where a method consists of A+B+C and embodiments where a method consists essentially of A+B+C. As described, the phrase “at least one of A, B, and C” is intended to include “at least one A or at least one B or at least one C,” and is also intended to include “at least one A and at least one B and at least one C.”

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples that follow serve to illustrate embodiments of the present disclosure without, however, being limiting in nature.

It will be apparent to those skilled in the art that various modifications and variations can be made in the compositions and methods according to the disclosure without departing from the spirit or scope of the invention. Thus, it is intended that the present invention cover the modifications and variations of this invention provided that they come within the scope of the appended claims and their equivalents.

### EXAMPLES

The following examples are intended to be non-limiting and explanatory in nature only.

### Example 1: Leave on Hair Compositions

The following compositions were prepared. All ingredients except the murumuru butter and nanocrystalline cellulose were premixed (CC1). The murumuru butter was heated until soft/melted and added to the premixture with continuous mixing. During the continuous mixing, the nanocrystalline cellulose (IC1) or cellulose (CC2) was added slowly to ensure even distribution in the mixture.

TABLE 1

| | INVENTIVE | COMPARATIVE | COMPARATIVE |
|---|---|---|---|
| | COMPOSITION | COMPOSITION | COMPOSITION |
| | 1 (IC1) | 1 (CC1) | 2 (CC2) |
| COMPONENT | (WT %) | (WT %) | (WT %) |
| BEHENTRIMONIUM | 0.474 | 0.474 | 0.474 |
| CHLORIDE | | | |
| CITRIC ACID | 0.01 | 0.01 | 0.01 |
| TRIDECETH-6 | 0.025 | 0.025 | 0.025 |
| CETRIMONIUM | 0.38 | 0.38 | 0.38 |
| CHLORIDE | | | |
| STEARYL ALCOHOL | 1 | 1 | 1 |
| CHLORHEXIDINE | 0.02 | 0.02 | 0.02 |
| DIHYDROCHLORIDE | | | |
| SODIUM BENZOATE | 0.2 | 0.2 | 0.2 |
| ISOPROPYL | 0.108 | 0.108 | 0.108 |
| ALCOHOL | | | |
| CETEARYL | 5 | 5 | 5 |
| ALCOHOL | | | |
| AMODIMETHICONE | 0.285 | 0.285 | 0.285 |
| MINERAL OIL | 1.5 | 1.5 | 1.5 |
| NANOCRYSTALLINE | 0.25 | — | — |
| CELLULOSE | | | |
| CELLULOSE | — | — | 0.25 |
| (hydroxyl propyl | | | |
| methyl cellulose, | | | |
| 2142) | | | |
| MURUMURU BUTTER | 1.25 | — | 1.25 |
| WATER | Q.S. | Q.S. | Q.S. |

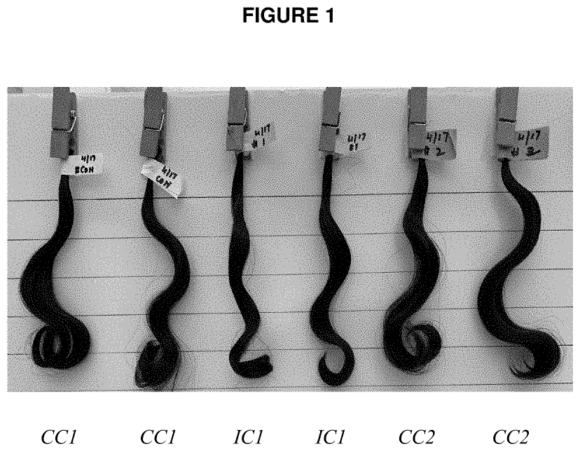

The compositions were applied to dry, curly hair, and the resulting curls evaluated as seen in FIG. 1. The curls treated with Inventive Composition 1 (IC1) were smooth, soft, defined curls, which retained their shape well, had very few flyaways, and had closed ends. The curls treated with Comparative Composition 1 (CC1) were smooth but did not retain the curl as well, and were frizzy at the ends. The curls treated with Comparative Composition 2 (CC2) were loose curls, had a powdery feel, were frizzy at the ends, and did not retain the curl as well. Surprisingly, Inventive Composition 1 (IC1) provided much better curl retention, smoothness, softness, and curl definition when compared to the comparative compositions (CC1 and CC2) which did not have the synergistic combination of nanocrystalline cellulose and murumuru butter.

### Example 2: Leave on Hair Compositions

The following compositions were prepared. All ingredients except the cellulose or nanocrystalline cellulose and the murumuru or shea butter were premixed (CC3). The murumuru (1C₂ and CC4) or shea butter (CC5) was heated until soft/melted and added to the premixture with continuous mixing. During the continuous mixing, the cellulose (CC4) or nanocrystalline cellulose (1C₂ and CC5) was added slowly to ensure even distribution in the mixture.

TABLE 2

| | INVENTIVE | COMPARATIVE | COMPARATIVE | COMPARATIVE |
|---|---|---|---|---|
| | COMPOSITION | COMPOSITION | COMPOSITION | COMPOSITION |
| | 2 (IC2) | 3 (CC3) | 4 (CC4) | 5 (CC5) |
| COMPONENT | (WT %) | (WT %) | (WT %) | (WT %) |
| BEHENTRIMONIUM | 0.474 | 0.474 | 0.474 | 0.474 |
| CHLORIDE | | | | |
| CITRIC ACID | 0.01 | 0.01 | 0.01 | 0.01 |
| TRIDECETH-6 | 0.025 | 0.025 | 0.025 | 0.025 |
| CETRIMONIUM | 0.38 | 0.38 | 0.38 | 0.38 |
| CHLORIDE | | | | |
| STEARYL ALCOHOL | 1 | 1 | 1 | 1 |
| CHLORHEXIDINE | 0.02 | 0.02 | 0.02 | 0.02 |
| DIHYDROCHLORIDE | | | | |
| SODIUM BENZOATE | 0.2 | 0.2 | 0.2 | 0.2 |
| ISOPROPYL | 0.108 | 0.108 | 0.108 | 0.108 |
| ALCOHOL | | | | |
| CETEARYL | 5 | 5 | 5 | 5 |
| ALCOHOL | | | | |
| AMODIMETHICONE | 0.285 | 0.285 | 0.285 | 0.285 |
| MINERAL OIL | 1.5 | 1.5 | 1.5 | 1.5 |
| NANOCRYSTALLINE | 0.25 | — | — | 0.25 |
| CELLULOSE | | | | |
| CELLULOSE | — | — | 0.25 | — |
| (hydroxyl propyl | | | | |
| methyl cellulose, | | | | |
| 2142) | | | | |
| MURUMURU BUTTER | 1.25 | — | 1.25 | — |
| SHEA BUTTER | — | — | — | 1.25 |
| WATER | Q.S. | Q.S. | Q.S. | Q.S. |

The compositions were applied to dry, curly hair, and the resulting curls evaluated. The curls treated with Inventive Composition 2 (IC2) were springy, smooth, soft, defined curls, which retained their shape well, and had closed ends. The curls treated with Comparative Composition 3 (CC3) were defined curls with a few flyaways and frizzy, open ends. The curls treated with Comparative Composition 4 (CC4) were loose curls, had a powdery feel, were frizzy at the ends, and did not retain the shape or curl as well. The curls treated with Comparative Composition 5 (CC5) were springy, retained the curl shape, and had closed ends, but had a greasy feel. Surprisingly, Inventive Composition 2 (IC2) provided much better curl retention, smoothness, softness, and curl definition, without a greasy feel, when compared to the comparative compositions (CC3, CC4, and CC5) which did not have the synergistic combination of nanocrystalline cellulose and murumuru butter.

### Example 3: Leave on Hair Compositions

Several additional inventive leave-on hair compositions were prepared as shown in Example 1, but with varying concentrations of nanocrystalline cellulose (0.25%, 0.5%, and 1%) and murumuru butter (1%, 1.25%, and 2%). These additional inventive compositions resulted in similar surprising improvement in curl retention, smoothness, softness, and curl definition when compared to the comparative compositions, demonstrating that the combination of nanocrystalline cellulose and murumuru butter provides synergistic results at alternate concentrations as well.

### Example 4: Additional Leave on Hair Composition Formulations

The following additional compositions may be prepared by any method, including the method described in Examples 1 and 2, or any other method known:

TABLE 3

| | ADDITIONAL | ADDITIONAL | ADDITIONAL |
|---|---|---|---|
| | COMPOSITION | COMPOSITION | COMPOSITION |
| | 1 (AC1) | 2 (AC2) | 3 (AC3) |
| COMPONENT | (WT %) | (WT %) | (WT %) |
| CATIONIC SURFACTANT | 0.1 | 5 | 10 |
| FATTY ALCOHOL | 0.1 | 10 | 15 |
| AMINOSILICONE | 0.1 | 5 | 10 |
| OIL | 0.1 | 5 | 10 |
| NANOCRYSTALLINE | 0.1 | 3 | 10 |
| CELLULOSE | | | |
| MURUMURU BUTTER | 0.1 | 5 | 10 |
| ADDITIONAL OPTIONAL | * | * | * |
| COMPONENTS (e g. | | | |
| preservatives, extracts, etc.) | | | |
| SOLVENT | Q.S. | Q.S. | Q.S. |

*optionally included in amounts as desired for each particular formulation

Leave-on compositions AC1-AC3 may be applied to wet or dry hair, and the hair optionally styled with or without heat.

1. A cosmetic composition comprising:
nanocrystalline cellulose;
murumuru butter;
at least one fatty alcohol;
at least one aminosilicone;
at least one oil; and
at least one cationic surfactant.

2. The cosmetic composition of claim 1, wherein the nanocrystalline cellulose is present in an amount ranging from about 0.01% to about 10%, relative to the total weight of the composition.

3. The cosmetic composition of claim 2, wherein the nanocrystalline cellulose is present in an amount ranging from about 0.01% to about 3%, relative to the total weight of the composition.

4. The cosmetic composition of claim 1, wherein the murumuru butter is present in an amount ranging from about 0.01% to about 10%, relative to the total weight of the composition.

5. The cosmetic composition of claim 4, wherein the murumuru butter is present in an amount ranging from about 0.1% to about 5%, relative to the total weight of the composition.

6. The cosmetic composition of claim 1, comprising nanocrystalline cellulose in an amount ranging from about 0.01% to about 3% and murumuru butter in an amount ranging from about 0.1% to about 5%, relative to the total weight of the composition.

7. The cosmetic composition of claim 1, wherein the at least one fatty alcohol is present in an amount ranging from about 0.1% to about 15%, relative to the total weight of the composition.

8. (canceled)

9. The cosmetic composition of claim 1, wherein the at least one aminosilicone is present in an amount ranging from about 0.1% to about 10%, relative to the total weight of the composition.

10. (canceled)

11. The cosmetic composition of claim 1, wherein the at least one oil is present in an amount ranging from about 0.01% to about 10%, relative to the total weight of the composition.

12. (canceled)

13. The cosmetic composition of claim 1, wherein the at least one cationic surfactant is present in an amount ranging from about 0.1% to about 10%, relative to the total weight of the composition.

14. (canceled)

15. A cosmetic composition comprising:
nanocrystalline cellulose in an amount ranging from about 0.01% to about 3%;
murumuru butter in an amount ranging from about 0.1% to about 5%;
at least one fatty alcohol in an amount ranging from about 3% to about 8%;
at least one aminosilicone in an amount ranging from 0.01% to about 2%;
at least one oil in an amount ranging from about 0.1% to about 3%; and
at least one cationic surfactant in an amount ranging from about 1% to about 5%,
all amounts relative to the total weight of the composition.

16. A method for treating the hair, the method comprising applying to the hair a composition comprising:
nanocrystalline cellulose;
murumuru butter;
at least one fatty alcohol;
at least one aminosilicone;
at least one oil; and
at least one cationic surfactant.

17. The method of claim 16, wherein the nanocrystalline cellulose is present in an amount ranging from about 0.01% to about 10%, relative to the total weight of the composition.

18. (canceled)

19. The method of claim 16, wherein the murumuru butter is present in an amount ranging from about 0.01% to about 10%, relative to the total weight of the composition.

20. (canceled)

21. The method of claim 16, comprising nanocrystalline cellulose in an amount ranging from about 0.01% to about 3% and murumuru butter in an amount ranging from about 0.1% to about 5%, relative to the total weight of the composition.

22. The method of claim 16, wherein the at least one fatty alcohol is present in an amount ranging from about 0.1% to about 15%, relative to the total weight of the composition.

23. (canceled)

24. The method of claim 16, wherein the at least one aminosilicone is present in an amount ranging from about 0.1% to about 10%, relative to the total weight of the composition.

25. (canceled)

26. The method of claim 16, wherein the at least one oil is present in an amount ranging from about 0.01% to about 10%, relative to the total weight of the composition.

27. (canceled)

28. The method of claim 16, wherein the at least one cationic surfactant is present in an amount ranging from about 0.1% to about 10%, relative to the total weight of the composition.

29. (canceled)

30. The method of claim 16, wherein the composition is a leave-in composition.

31. (canceled)

32. (canceled)
